(19) United States
(12) Patent Application Publication
(10) Pub. No.: US 2011/0008203 A1
(43) Pub. Date: Jan. 13, 2011

(54) Vehicle scent dispenser

(75) Inventor: Steve Morgan, Vancouver, WA (US)

Correspondence Address: ROBERT IRELAND, P.O. BOX 273, BANKS, OR, 97124, US

(73) Assignee: 'LiL' Stinky LLC, Vancouver, WA (US)

(21) Appl. No.: 12/803,154

(22) Filed: Jun. 21, 2010

Related U.S. Application Data
(60) Provisional application No. 61/270,491, filed on Jul. 8, 2009.

(51) Int. Cl. A61L 9/14; B01D 50/00

(52) U.S. Cl. 422/5; 261/61; 96/222; 96/108

(57) ABSTRACT

A system and method for delivering multiple bursts of stinky air using a scent dispensing system is disclosed. A scent dispensing system comprising a scent chamber, liquid scent, a trigger switch, a positive pressured air source, a first one-way valve, and a second one-way valve. The operation of the system begins with loading the liquid scent into the scent chamber and sealing it, then activating the trigger switch causing the positive pressured air source to direct incoming air to the first one-way valve, the first one-way valve opens upon the incoming air reaching a desired positive pressure flowing incoming air through the first one-way valve into the scent chamber, the scent chamber combines the incoming air with the liquid scent thereby producing stinky air, the second one-way valve opens upon the pressure in the scent chamber reaching a desired positive pressure, thereby flowing the pressurized stinky air through the second one-way valve and out of the system, away from the vehicle. Upon deactivating the trigger switch, the first and second one-way valves close, sealing the scent chamber and the stinky air within from both the inside of the vehicle and outside the vehicle, leaving the system ready to release another burst upon activating the trigger switch.

### BACKGROUND OF THE INVENTION

1. Technical Field of the Invention

The present invention relates in general to delivery of scented air. More specifically, the present invention relates to a system and method for controlled delivery of scented air to a localized target.

2. Description of the Related Art

There are instances while in a vehicle that a user would benefit from releasing scented air in order to communicate, attract, or repel people or animals. For instance, if an unwanted bear approached a user's vehicle, the user would benefit from releasing a scent that repelled said bear. The same could be true if a hunter wanted to disguise the smell of his vehicle, a masking scent released on demand would be beneficial. The problems with releasing scent from a moving vehicle are many, the most obvious being unwanted collateral scent escaping into the user's vehicle. Another problem of concern is the lingering release when the vehicle is parked within a confined space. The types of scent that are affective on animals are usually unpleasant scents that the user would not want tainting their vehicle or the inside of their garages.

Drivers on the road or in traffic could also benefit from a method to communicate directly with other drivers in such a manner that the message is understood. For example, a driver who is being tailgated may want to signal that the close proximity of the following car is not appreciated. Many methods for the drivers to deliver appropriate message directly with other drivers have been developed in the art such as sign language, oral exchanges, or honking, but nothing in the form of scent release which would affect one of the most responsive and immediate senses, the sense of smell.

Most conventional scent releasing systems involve squirt or spray technology wherein the accuracy of the squirt or spray provides the delivery to a target-specific area. This conventional squirt or spray method may be unlawful in a “driving down the road” application, as the scent liquid could blur windshields causing vision degradation. Hence this method is not acceptable. Another drawback of the conventional squirt method is that the scent projected from the scent releasing system will stink out the triggering vehicle and the scent target.

Therefore, as can be appreciated, there is a need of system and method that would provide an on demand controlled burst of scent capable of being repeatedly expelled from a stopped or moving vehicle. Further, such a needed system and method would propel a scent without liquid projectile, and without stinking out the triggering vehicle. Moreover, such a system and method would stop the stinking effect after the user quits releasing the scent thus preventing unwanted odor in a garage or other enclosed area.

### SUMMARY OF THE INVENTION

To minimize the limitations found in the prior art, and to minimize other limitations that will be apparent upon the reading of the specifications, the present invention provides a scent dispensing system.

The scent dispensing system comprises a user first loading scent liquid into a scent chamber and sealing it, making it ready for use, then when ready to dispense stinky air, triggering an air source to flow incoming air into a sealed system; the incoming air is in flow communication with a first one-way valve that opens once the incoming air reaches a desired positive pressure, once open, the incoming air flows through the first one-way valve entering the scent chamber, the scent chamber contains liquid scent that evaporates into the incoming air making stinky air, the scent chamber is also in flow communication with a second one-way valve that opens upon the scent chamber reaching a desired positive pressure, once open, the stinky air blasts through the second one-way valve at the target. The scent chamber is of adequate volume to provide ample incoming air to combine with the scent vapors, thereby increasing the scent affect when released. After deactivating the system, the scent chamber seals away the stinky air, and is ready for the next activation, as one loading of scent liquid will provide multiple activations without needing reload. The scent dispensing system described herein is applicable to install in a plethora of vehicles such as cars, trucks, all terrain vehicles, motorcycles, or bicycles.

In another aspect of the present invention, a method for delivering stinky air using the scent dispensing system. In the method, the scent oil or scent liquid is poured into the scent chamber through at least one load hole in the scent chamber and then sealingly closed. A triggering mechanism is activated to introduce incoming air into the scent dispensing system. Upon triggering, the incoming air comes in flow communication with a first one-way valve and upon the incoming air reaching a desired positive pressure, the one-way valve opens, allowing for the positive pressured incoming air to flow into the scent chamber. Once in the scent chamber the incoming air impacts and combines with the scent liquid. The now stinky air comes into flow communication with the second one-way valve and upon the stinky air reaching a desired positive pressure, opens allowing a flow or burst of stinky air out and away from the vehicle. Upon un-triggering or shutting off the incoming air the first one-way valve and the second one-way valve close, sealingly isolating the scent chamber from the vehicle and outside air, while being prepared to trigger again.

One objective of the invention is to provide a system wherein a user in a vehicle can trigger a release of scent from a vehicle, in multiple bursts without scent reload, and without causing the scent to be inadvertently leaked either in or out of the vehicle between bursts.

Another objective of the invention is to provide a system wherein passing air outside a moving vehicle is captured by an air funnel that directs the now pressured air into the scent dispensing system.

Another objective of the invention is to provide an improved scent chamber benefitting from internal structure that disrupts the laminar flow of incoming air to improve liquid scent vaporization and combination with the incoming air.

Still another objective of the invention is to provide at least one absorption pad within the scent chamber capable of absorbing scent liquid and then evaporating the same into the incoming air.

It is also the objective of the invention to provide a scent chamber having at least one load hole positioned gravitationally above at least one absorption pad within the scent chamber for easy loading the system with scent liquid, thereby providing multiple bursts without reloading.

A final objective of the invention is to provide a scent chamber that not only sealingly holds the scent liquid and stinky air with a one-way valve on each end, but also stretches or elastically expands with the introduction of positive pressure thereby increasing the volume of stinky air with constant positive pressure for blasting out the stinky air out upon the second one-way valve opening.

These and other advantages and features of the present invention are described with specificity so as to make the present invention understandable to one of ordinary skill in the art.

### BRIEF DESCRIPTION OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention, thus the drawings are generalized in form in the interest of clarity and conciseness.

FIG. 1 is a diagrammatic representation of the present invention, illustrating a scent dispensing system;

FIG. 2 is a sectional view taken along lines A-A′ and B-B′ in FIG. 1 of the present invention;

FIG. 3 is a close-up view of a sealing technique utilized by the second one-way valve of the present invention;

FIG. 4 is a diagrammatic representation of the present invention, illustrating the scent dispensing system in use;

FIG. 5a is a diagrammatic representation of another embodiment of the present invention, illustrating the scent dispensing system without an air blower;

FIG. 5b is a blow-up view of the scent dispensing system without an air blower shown in FIG. 5a;

FIG. 6 is a front view of a disruption structure within the scent chamber of the present invention;

FIG. 7 is a diagrammatic representation of yet another embodiment of the present invention, illustrating the air blower configured to operate with an internal solenoid;

FIG. 8 is a cross sectional view of the present invention, illustrating the scent chamber with a pair of end caps;

FIG. 9a is a side view of a scent dispenser of the scent chamber of the present invention;

FIG. 9b is a side view of a scent absorber of the scent chamber of the present invention;

FIG. 9c is a cross sectional view of a post-filter of the present invention; and

FIG. 10 is a top plan view of a plurality of absorption pads on the scent dispenser of the present invention.

### DETAILED DESCRIPTION OF THE DRAWINGS

In the following discussion that addresses a number of embodiments and applications of the present invention, reference is made to the accompanying drawings that form a part of hereof, and in which is shown by way of illustration specific embodiments in which the invention may be practiced. It is to be understood that other embodiments may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

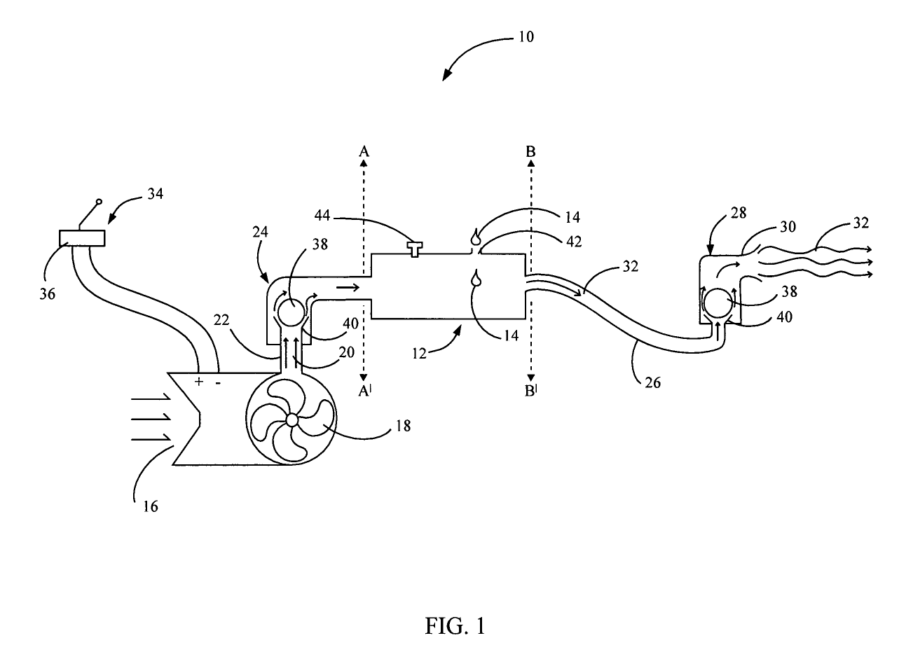

FIG. 1 is a schematic representation of a scent dispensing system 10. The scent dispensing system 10 comprises a scent chamber 12 designed to store a scent oil 14. An air blower 18 to receive external air 16 and to facilitate a flow of positive pressured air 20 towards the scent chamber 12 through a first tube 22 connected with the air blower 18, a first one way-valve 24 that allows the positive pressured air 20 to flow in one direction towards the scent chamber 12 and a second tube 26 that allows the positive pressured stinky air 32 to flow towards a release horn 30 through a second one way valve 28 connected with the second tube 26. The second one way valve 28 opens after the stinky air 32 reaches a desired pressure, and closes immediately after the air blower 18 shuts off.

By definition, a one-way valve as shown and described as a first one-way valve 24 and second one-way valve 28 only communicate air in one direction, thereby sealing against air flowing in the opposing direction. As such, these one-way valves are flow directional by function, and when installed, must be installed accordingly. Some or all of the one-way valves described herein open only in the flow direction after being impacted by a set desired positive pressure, thereby being sealed in both directions until the flow direction pressure reaches the set positive pressure of the valve. Also, one-way valves of the sort utilized by the present invention close, when the positive pressure falls below the opening pressure.

One skilled in the art would understand how the electrical power would be applied to the air blower 18 which is turned on and off by a trigger switch 36, and therefore to improve brevity in this specification is mentioned as a known art but not described in detail herein.

The scent chamber 12 evaporates the scent oil 14 by combining the positive pressured air 20 with the scent oil 14 to generate the positive pressured stinky air 32 and directs a controlled amount of the positive pressured stinky air 32 along the second tube 26 towards the second one-way valve 28 that propels the controlled amount of the positive pressured stinky air 32 to at least one target (not shown) and in this embodiment through a release horn 30. The scent dispensing system 10 is designed to install in a vehicle (not shown) and the at least one target (not shown) may be an animal or a tailgating vehicle.

The scent chamber 12 may be a cylinder with caps, a one continuous plastic injection molded piece, or an elastic bladder that increases in size as pressure and volume build within. The advantage of the elastic or pliable scent chamber 12 is the amount of stinky air that could be released at once in one long continuous burst. To further augments a scent chamber 12 having this elastic or pliable feature would be a first one-way valve 24 that opens at a lower pressure than the second one-way valve 28. In this configuration the scent chamber 12 builds pressure and volume as held back by the second one-way valve 28 until it reaches it's opening pressure. Once open, the outflow of stinky air 32 is thereby increased in those applications where it is desired.

The air blower 18 requires activation of a triggering mechanism 34 to facilitate the flow of the positive pressured air 20 towards the scent chamber 12. In the preferred embodiment the triggering mechanism 34 is a trigger switch 36. When the trigger switch 36 is on, the air blower 18 gets activated and pushes the external air 16 towards the first one-way valve 24 through the first tube 22. The first one-way valve 24 utilizes a sealing technique that allows the positive pressured air 20 to flow in one direction towards the scent chamber 12. In the preferred embodiment, the sealing technique comprises a ball 38 and a seat 40. The positive pressured air 20 lifts the ball 38 off of the seat 40 providing an opening to flow towards the scent chamber 12. The system 10 is adaptable to operate with other sealing techniques like spring held valves, needle and seat valves, flapper valves, knife valves, and rotational ball valves. The positive pressured air 20 combines with the scent oil 14 present in the scent chamber 12 to form the positive pressured stinky air 32. The positive pressured stinky air 32 flows out from the scent chamber 12 towards the second one-way valve 28 through the second tube 26. The positive pressured stinky air 32 pushes the ball 38 off of the seat 40 providing an opening to flow towards the at least one target (not shown). Depending on application, the release towards the target may be enhanced through a release horn 30.

In the preferred embodiment, the scent chamber 12 includes at least one load hole 42 for pouring the scent oil 14 into the scent chamber 12 and at least one load hole 42 are closed by means of a closure means 44 like plug. The system 10 is adaptable to use other methods of pouring the scent oil 14 like injection and spraying.

In the preferred embodiment, to further protect against stinky air 32 from flowing back into the vehicle, the first one-way valve 24 opens at a great positive pressure than the second one-way valve 28, so that upon shut off of the pressured air 20, the first one-way valve 24 positively closes before the second one-way valve 28, thereby preventing any back flow of stinky air 32 into the vehicle.

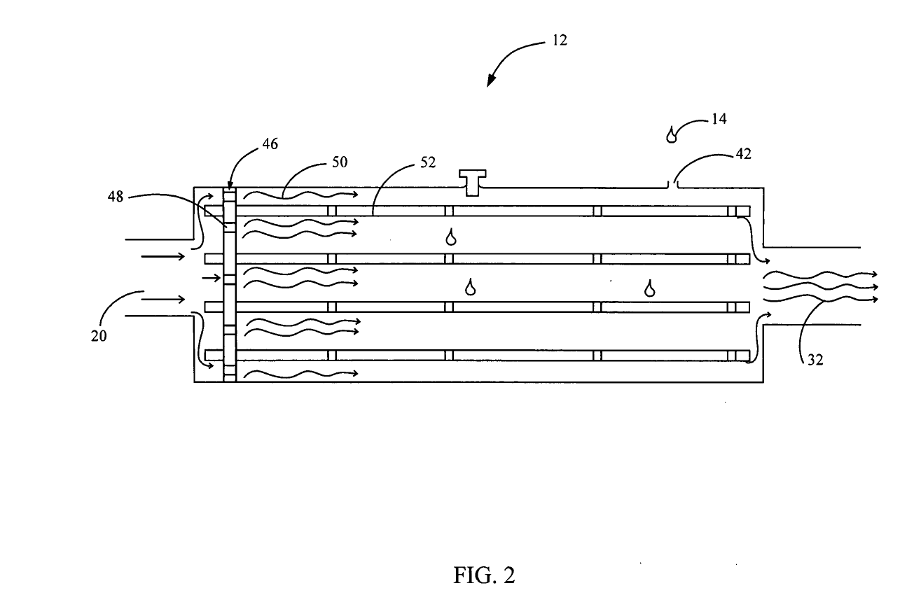

FIG. 2 is a sectional view taken along lines A-A′ and B-B′ in FIG. 1 of the present invention. When the positive pressured air 20 moves past the sealing technique and enters the scent chamber 12, the positive pressured air 20 impacts with a disruption structure 46 having a plurality of apertures 48 that disrupts a laminar flow of the positive pressured air 20 causing turbulence within the positive pressured air 20 resulting in disrupted air 50 within the scent chamber 12. The disrupted air 50 improves the vaporization and combination of the positive pressured air 20 with the scent oil 14 over an undisrupted air flow. The scent chamber 12 includes at least one absorption pad 52 adaptable to absorb the scent oil 14 and to enhance the evaporation of the scent oil 14 for generating scent enhanced air within the scent chamber 12. Dropping the scent oil 14 through the plurality of load holes 42 allows for gravity feed of the scent oil 14 into the absorption pads 52. The disrupted air 50 flows through and around the absorption pads 52. At least one absorption pad 52 may be constructed from material selected from a group consisting of felt and/or sponge and may be vertically or horizontally disposed within the scent chamber 12. The scent enhanced air is in a vaporized form without any liquid content and flows towards the second tube 26 as the positive pressured stinky air 32.

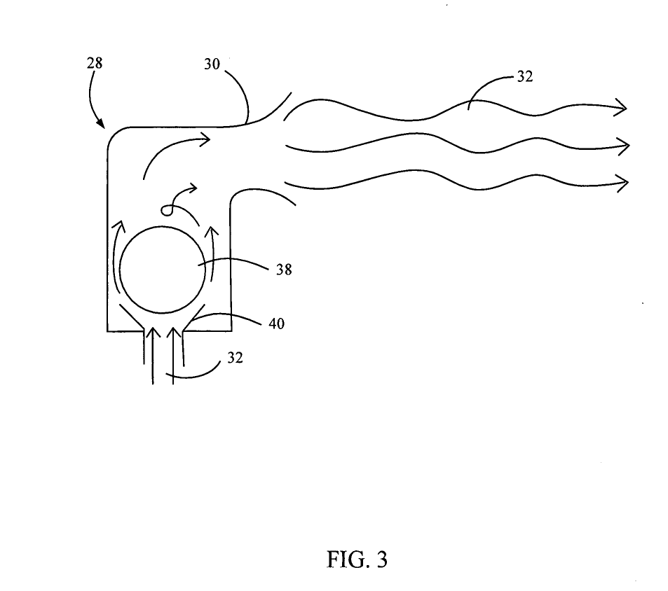

FIG. 3 is a close-up view of the sealing technique utilized by the second one-way valve 28 for sealing the scent chamber 12 to prevent leakage of the scent oil 14. The first one-way valve 24 and the second one-way valve 28 utilize the sealing technique to open only in one direction. When the positive pressured stinky air 32 moves past the second tube 26 and enters into the second one-way valve 28, the positive pressured stinky air 32 pushes the ball 38 off of the seat 40 providing an opening to flow around the ball 38 and towards the release horn 30 to impact with the at least one target (not shown). The first one-way valve 24 and the second one-way valve 28 open upon a desired positive pressure and sealingly close when the pressure reduces below the “open” pressure. One advantage of the ball 38 and the seat 40 in the sealing technique is that if the scent oil 14 makes its way into the second tube 26, the flow of positive pressured stinky air 32 is allowed to pass through the space between the ball 38 and the seat 40, but the scent oil 14 would be unable to. A further advantage of the ball 38 and the seat 40 is that shape of the ball 38 naturally causes turbulence on the back side which in turn helps keep the positive pressured stinky air 32 combined with the scent oil 14 in vapor suspension.

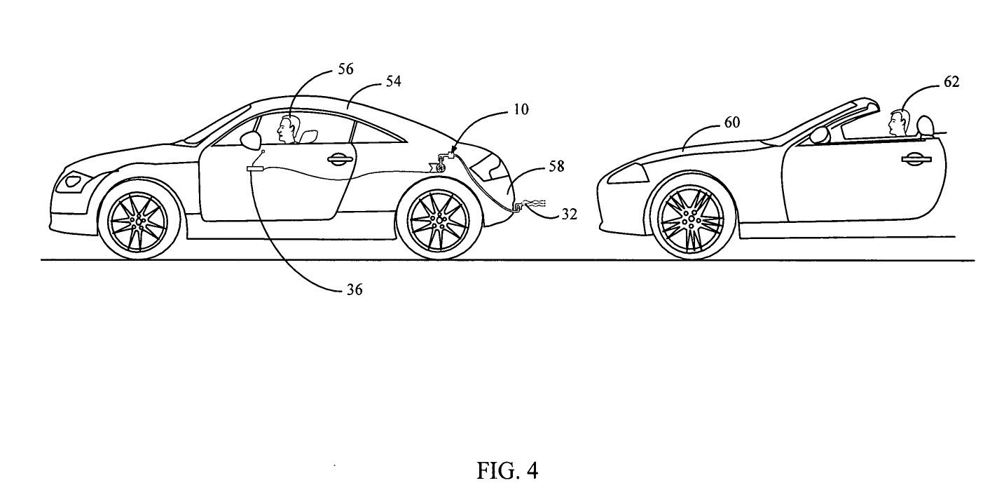

FIG. 4 is a diagrammatic representation of the scent dispensing system 10 in use. In FIG. 4, a car 54 is dangerously followed by a tailgating car 60. A user 56 in the car 54 pulls the trigger switch 36 into an on position that energizes the air blower 18 and allows the positive pressured air 20 to flow through the scent chamber 12 and towards the release horn 30 as the positive pressured stinky air 32. The positive pressured stinky air 32 flows from the rear part 58 of the car 54 and impacts with the tailgating car 60 having desired communication with at least one occupant 62 in the tailgating car 60. In this way the occupant 62 in the tailgating car 60 gets informed about the possible dangerous-driving-too-close situation.

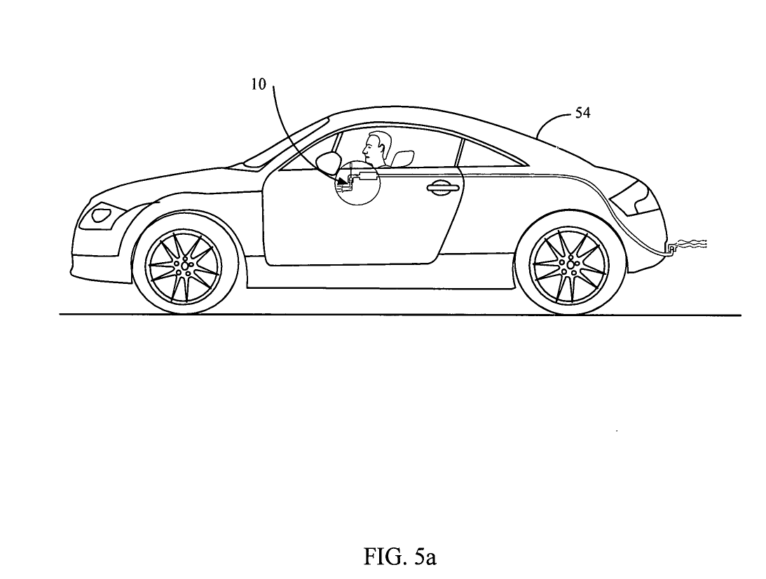

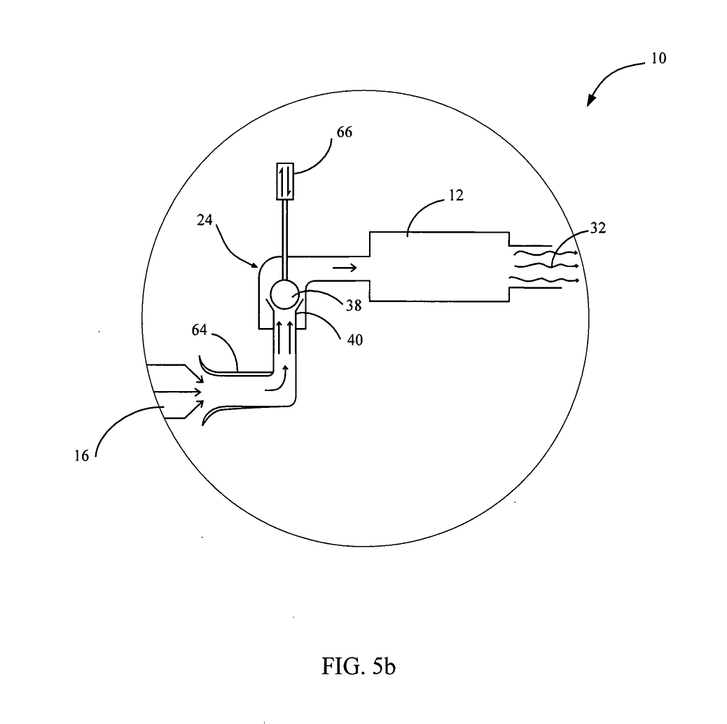

FIG. 5a is a diagrammatic representation of another embodiment of the present invention, illustrating the scent dispensing system 10 without an air blower 18. In this embodiment, the force required to move the external air 16 into the scent chamber 12 is accomplished by the velocity of the car 54. FIG. 5b is a blow-up view of the scent dispensing system without an air blower shown in FIG. 5a. An air funnel 64 directs the external air 16 towards the first one-way valve 24 and the sealing technique comprising the ball 38 and the seat 40 is augmented by an actuation shaft 66. The actuation shaft 66 can be manually lifted for lifting the ball 38 at the user's desire thus introducing the external air 16 around the ball 38 and towards the scent chamber 12.

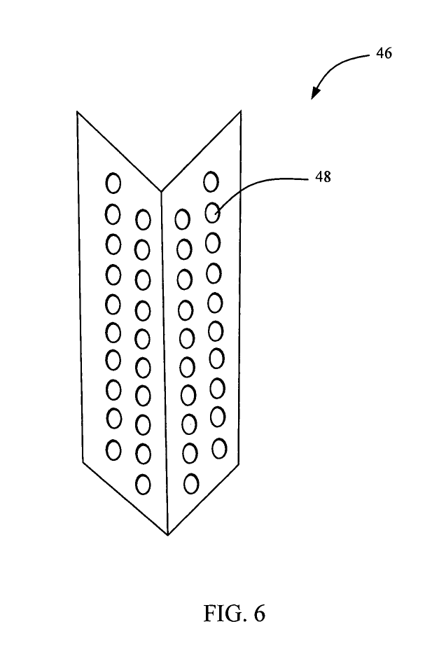

FIG. 6 is a front view of the disruption structure 46 within the scent chamber 12. The disruption structure 46 can be easily constructed from an angle run of metal having air apertures 48 which can be in any configuration adaptable to disrupt flow of the positive pressured air 20, thereby enhancing the vaporization of the scent oil 14 out of the absorption pads 52.

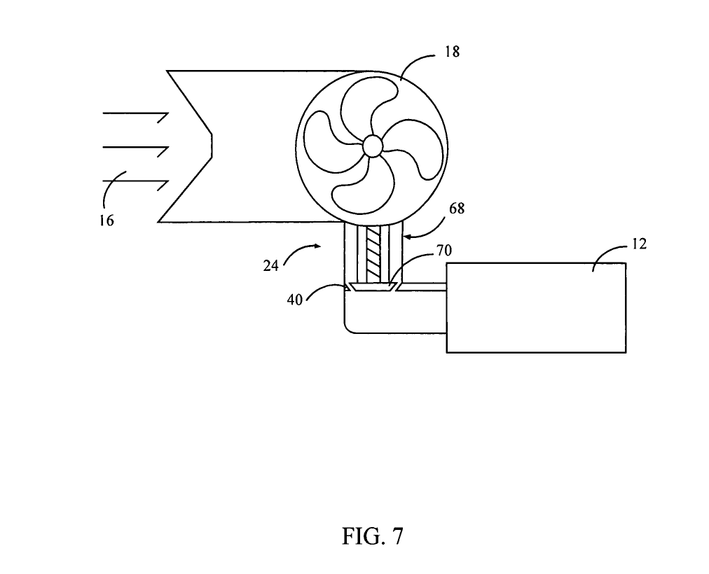

FIG. 7 is a diagrammatic representation of yet another embodiment of the present invention, illustrating the air blower 18 configured to operate with an internal solenoid 68. In this embodiment, the sealing technique utilized by the first one-way valve 24 comprises the seat 40 and a plunger 70 compressed into the seat 40. The user 56 utilizes the trigger switch 36 to open the internal solenoid 68 contemporaneously with the powering up of the air blower 18.

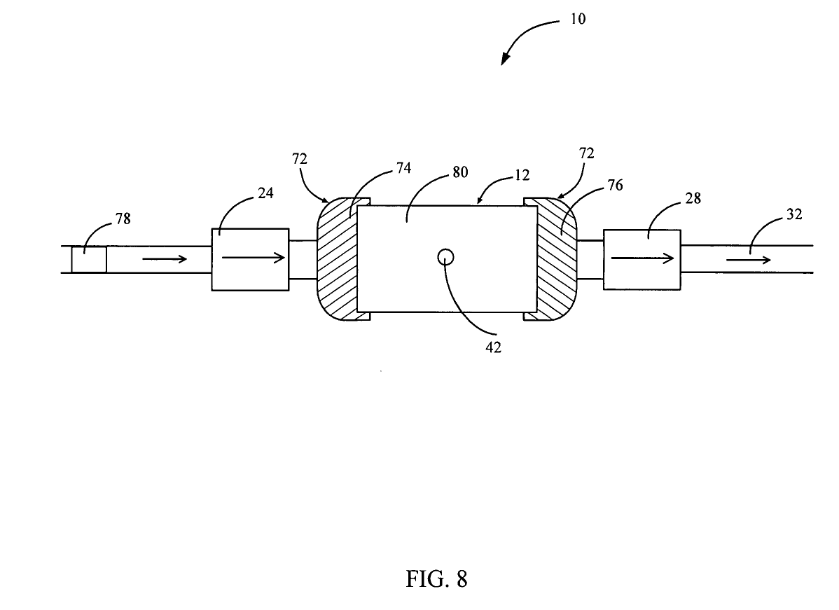

FIG. 8 is a cross sectional view of the present invention, illustrating the scent chamber 12 with a pair of end caps 72. The scent chamber is constructed from a cylindrical tube and includes a pair of hollow sides sealed with the pair of end caps 72 which includes a first end cap 74 and a second end cap 76. The scent dispensing system 10 includes a pre-filter 78 located before the first one-way valve 24 to remove particulates present in the positive pressured air 20. The positive pressured air 20 is put into the pre-filter 78 and after filtration the positive pressured air 20 is pushed towards a main body 80 of the scent chamber 12. Inside the main body 80 of the scent chamber 12, the positive pressured air 20 combines with the scent oil 14 that is filled and refilled into the main body 80 through at least one load hole 42. The positive pressured air 20 turns into the positive pressured stinky air 32 and leaves the scent chamber 12 through the second one-way valve 28 and expels out of the rear part 58 of the car 54 towards the tailgating car 60.

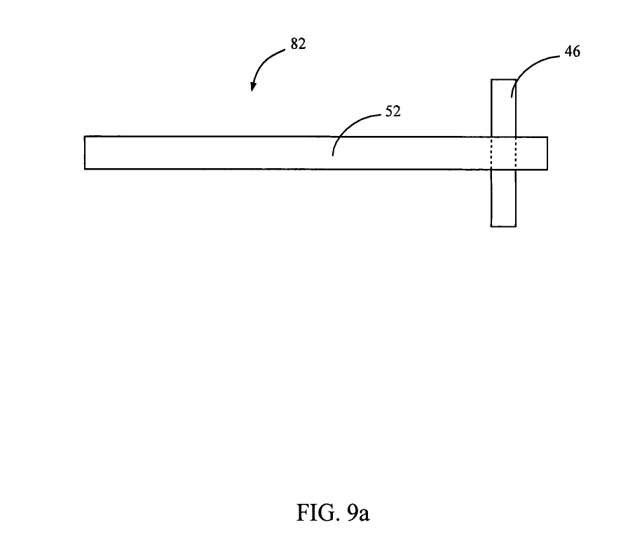

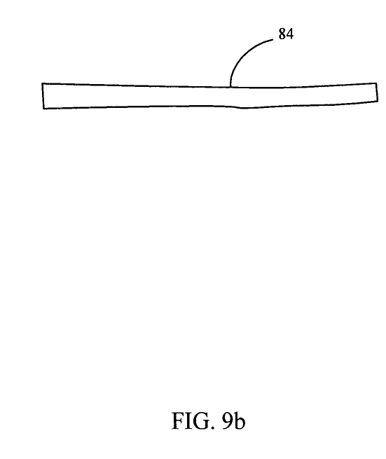

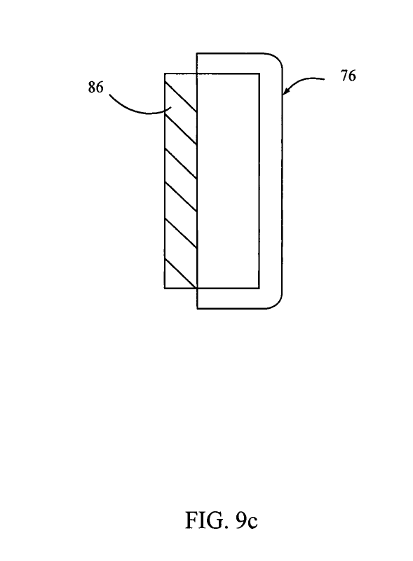

FIG. 9a is a side view of a scent dispenser 82 located inside the scent chamber 12 of the present invention. The scent dispenser 82 includes at least one absorption pads 52 and the disruption structure 46. FIG. 9b is a side view of a scent absorber 84 located inside the scent chamber 12 of the scent dispensing system 10. When the positive pressured air 20 enters into the scent chamber 12, it comes into contact with the scent dispenser 82. The scent absorber 84 collects excess scent in the scent chamber 12 and is placed at the bottom portion of the main body 80 of the scent chamber 12. FIG. 9c is a cross sectional view of a post-filter 86 located between the scent chamber 12 and the second one-way valve 28 to remove particulates present in the positive pressured stinky air 32.

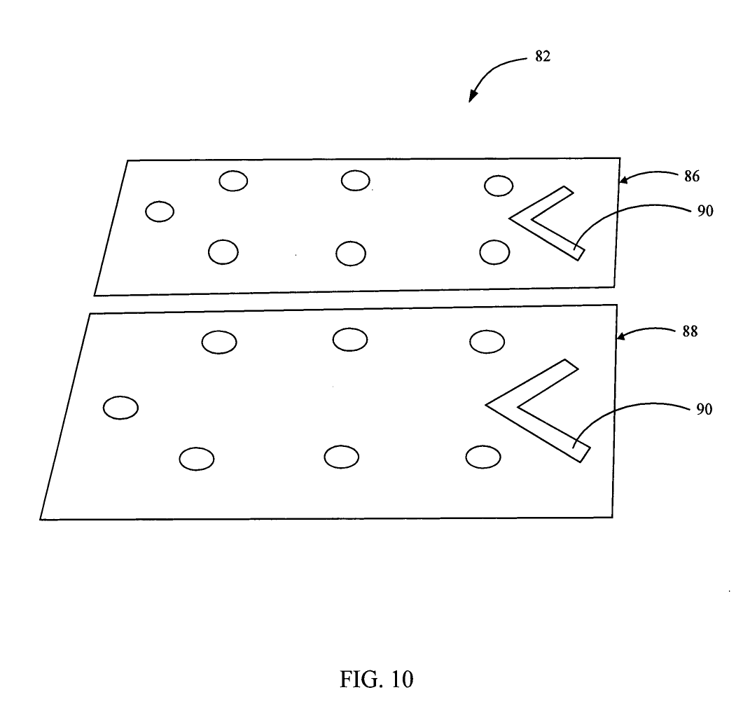

FIG. 10 shows a top plan view of the absorption pads 52 on the scent dispenser 82. The plurality of absorption pads 52 includes a liquid dispenser 86 and a disrupter plate 88 on which the liquid dispenser 86 is placed. The liquid dispenser 86 and the disrupter plate 88 include an opening 90 for inserting the disruption structure 46. When the positive pressured air 20 is pushed into the main body 80, it first hits the disruption structure 46 creating a turbulent air and the positive pressured air 20 is allowed to pass through the liquid dispenser 86 which lies on top of the disrupter plate 88 to create an aerosol without allowing the scent oil 14 to escape from the scent chamber 12.

The preferred embodiment includes a method for delivering scented air using the scent dispensing system 10. In the method, the scent oil 14 is poured into the scent chamber 12 through at least one load hole 42 in the scent chamber 12. A triggering mechanism 34 is activated to energize the air blower 18 in the scent dispensing system 10. Upon activation, the air blower 18 receives the external air 16 and facilitates the flow of the positive pressured air 20 towards the scent chamber 12 through the first tube 22 connected with the air blower 18. The positive pressured air 20 is directed towards the scent chamber 12 by the first one-way valve 24 and is impacted with a disruption structure 46 in the scent chamber 12 causing turbulence within the positive pressured air 20. The scent oil 14 is evaporated in the scent chamber 12 by combining the scent oil 14 with the positive pressured air 20 using the plurality of absorption pads 52 to generate the positive pressured stinky air 32. A controlled amount of the positive pressured stinky air 32 is directed towards a second one-way valve 28 along the second tube 26 connected with the scent chamber 12 and propelled towards the at least one target 60 through the release horn 30.

The foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention not be limited by this detailed description, but by the claims and the equivalents to the claims appended hereto.

What is claimed is:

1. A scent dispensing system comprising:
Liquid scent;
A trigger switch;
A positive pressured air source;
A first one-way valve;
A scent chamber; and
A second one-way valve;
The liquid scent is poured into the scent chamber and sealed;
Upon activating the trigger switch, the positive pressured air source causes incoming air to be directed to the first one-way valve;
The first one-way valve opens upon the incoming air reaching a positive pressure, thereby flowing incoming air into the scent chamber;
The scent chamber combines the incoming air with the liquid scent, providing stinky air;
The second one-way valve opens upon the pressure in the scent chamber reaching positive pressure, thereby flowing the pressurized stinky air out and at the target.

2. A scent dispensing system according to claim 1, further comprising a pre-filter located in the downstream air, before the first one-way valve.

3. A scent dispensing system according to claim 1, further comprising a post-filter located after the scent chamber, before the second one-way valve.

4. A scent dispensing system according to claim 1, further comprising a scent chamber, benefitting from a disruption structure positioned in the flow of incoming air within the scent chamber, arranged to improve the combination of the liquid scent with the incoming air.

5. The scent dispensing system of claim 1 wherein the first one-way valve or second one-way valve may be selected from a group consisting of ball and seat valves, spring held valves, needle and seat valves, flapper valves, knife valves, and rotational ball valves.

6. A scent dispensing system according to claim 1, further comprising a scent chamber, benefitting from a disruption structure having at least one layer of absorption material on at least one side.

7. A scent dispensing system according to claim 1, further comprising an absorbing filter, located within the scent chamber, arranged to have the scented air flow through the absorbing filter prior to exiting the scent chamber.

8. The scent dispensing system of claim 1 wherein the first one-way valve opens at a greater positive pressure that the second one-way valve.

9. The scent dispensing system of claim 1 wherein the scent chamber includes a disruption structure having a plurality of apertures that disrupts the laminar flow of the positive pressured air, positioned to cause turbulence within the scent chamber.

10. The scent dispensing system of claim 1 wherein the scent chamber further includes at least one absorption pad, adaptable to absorb and hold a quantity of the scent liquid adapted to increase the number of bursts of stinky air before reloading scent liquid into the scent chamber.

11. The scent dispensing system of claim 1 wherein the scent chamber is constructed of pliable material arranged to allow expansion upon introduction of positive pressured air.

12. The scent dispensing system of claim 8 wherein the scent chamber further includes at least one load hole constructed within the scent chamber, located gravitationally above the absorption pad.

13. A method for delivering multiple bursts of stinky air using a scent dispensing system, the method comprising the steps:
(a) providing the scent dispensing system and pouring the scent liquid into the scent chamber through at least one load hole and sealing the scent chamber;
(b) activating a triggering mechanism to turn on air source that provides positive pressured air to a first one-way valve;
(c) upon reaching a desired positive pressure, the first one-way valve opens and flows the air into a scent chamber;
(d) the incoming air combines with the scent liquid within the scent chamber making for stinky air;
(e) upon the scent chamber reaching a desired positive pressure, the second one-way valve opens and flows the stinky air out of the scent dispensing system at the target;
(f) deactivating the triggering mechanism causing the incoming pressured air to drop, causing first one-way valve to sealingly close;
(g) upon the first one-way valve sealingly closing, the pressure in the scent chamber drops, and the second one-way valve sealingly closes; and
(h) activating a triggering mechanism to turn on the air source that causes a burst of stinky air as described above.

14. A method for delivering multiple bursts of stinky air using a scent dispensing system according to claim 13, further comprising a step of positioning a disruption structure within the scent chamber located to improve the combination of the liquid scent with the incoming air.

15. A method for delivering multiple bursts of stinky air using a scent dispensing system according to claim 13, further comprising a step of positioning a disruption structure having at least one layer of absorption material on at least one side within the scent chamber located to improve the combination of the liquid scent with the incoming air.

16. A method for delivering multiple bursts of stinky air using a scent dispensing system according to claim 13, wherein the first one-way valve opens at a greater positive pressure that the second one-way valve.

17. A method for delivering multiple bursts of stinky air using a scent dispensing system according to claim 13, further comprising a step of positioning a disruption structure having a plurality of apertures that disrupts the laminar flow of the positive pressured air, positioned to cause turbulence within the scent chamber.

18. A method for delivering multiple bursts of stinky air using a scent dispensing system according to claim 13, further comprising a step of positioning at least one absorption pad, adaptable to absorb and hold a quantity of the scent liquid adapted to increase the number of bursts of stinky air before reloading scent liquid into the scent chamber.

19. A method for delivering multiple bursts of stinky air using a scent dispensing system according to claim 13, further comprising a step of constructing the scent chamber from a pliable material arranged to allow expansion upon introduction of positive pressured air.
